(19) United States
(12) Patent Application Publication
(10) Pub. No.: US 2026/0053455 A1
(43) Pub. Date: Feb. 26, 2026

(54) STAND COLUMN ASSEMBLY FOR X-RAY IMAGING SYSTEM AND X-RAY IMAGING SYSTEM

(71) Applicant: GE Precision Healthcare LLC, Waukesha, WI (US)

(72) Inventors: Bin Liu, Beijing (CN); Bingkun Xue, Beijing (CN); Yuqing Li, Beijing (CN); Fusheng Li, Beijing (CN)

(21) Appl. No.: 19/306,578

(22) Filed: Aug. 21, 2025

(30) Foreign Application Priority Data
Aug. 24, 2024 (CN) 202411188382.5

(51) Int. Cl. A61B 6/00; A61B 6/04; A61B 6/06

(52) U.S. Cl. CPC A61B 6/4452; A61B 6/0407; A61B 6/0487; A61B 6/06; A61B 6/4464; A61B 6/547; A61B 6/56

(57) ABSTRACT

Provided in the present application are a stand column assembly for an X-ray imaging system and an X-ray imaging system. The stand column assembly includes a base and a stand column that are assembled together. The base is configured to be disposed on a horizontal supporting surface and stationary relative to the supporting surface, and is configured to support a subject to be imaged. The stand column includes a main body portion and an X-ray detector, the main body portion being rotatably connected to the base and being erected opposite the supporting surface, and the X-ray detector being mounted on the main body portion. Further, there exists an axis of rotation perpendicular to the supporting surface and passing through the base, and the main body portion is rotatable about the axis of rotation relative to the base within a predetermined range.

### CROSS-REFERENCE TO RELATED APPLICATIONS

This application is based on and claims priority to Chinese Application No. 202411188382.5, filed on Aug. 28, 2024, the entire contents of which is herein incorporated by reference.

### TECHNICAL FIELD

The present application relates to the field of imaging, and more specifically to a stand column assembly for an X-ray imaging system and an X-ray imaging system including the stand column assembly.

### BACKGROUND

In an X-ray imaging system, X-rays from an X-ray generator are directed towards a subject to be imaged to achieve imaging, the subject to be imaged typically being a patient in a medical diagnostic application.

FIG. 27 shows a structure of an X-ray imaging system. As shown in FIG. 27, the X-ray imaging system includes a suspension apparatus 101, an X-ray generation mechanism 201, a stand column assembly 301, and an examination table 401. The suspension apparatus 101 may be mounted on a ceiling or a wall of a building, etc. The suspension apparatus 101 may include a main body frame 111, a telescopic cylinder 121 and an angle adjustment mechanism 131 that are assembled together, such that the suspension apparatus 101 can move within a predetermined range in multiple spatial degrees of freedom, and further the suspension apparatus 101 may be utilized to hold the X-ray generation mechanism 201 and adjust the position and posture of the X-ray generation mechanism 201. The X-ray generation mechanism 201 may include an X-ray tube generator and an X-ray collimator, the X-ray tube generator being configured to generate X-rays, and the X-ray collimator being configured to confine the X-rays generated by the X-ray tube generator within a predetermined range.

Further, the stand column assembly 301 or the examination table 401 is used according to sites of the subject to be imaged that need to be imaged and the state of the subject to be imaged itself, where the subject to be imaged may lie on the examination table 401 or stand in front of the stand column assembly 301. After the subject to be imaged lies down or stands in position, the X-rays generated by the X-ray tube generator and collimated by the X-ray collimator penetrate a predetermined site of the subject to be imaged. Then, for example, an X-ray detector provided at the stand column assembly 301 and the examination table 401 detects X-rays penetrating the subject to be imaged, the X-ray detector generates an output signal based on the intensity of rays impacting each discrete region of the detector, and processes the output signal to generate an image that can be displayed for inspection, and the image can be displayed in a display apparatus of the X-ray imaging system. Thus, by utilizing the difference in the penetration capability of X-rays to different substances, the X-rays penetrating the subject to be imaged are detected and processed to finally obtain an image showing the internal configuration of the subject to be imaged.

However, in such an X-ray imaging system, the stand column assembly 301 is stationary after being mounted on a floor of a building. Therefore, although the position and posture of the X-ray generation mechanism 201 can be adjusted by the suspension apparatus 101 such that the X-ray generation mechanism 201 can rotate around the subject to be imaged, the position of the X-ray detector remains unchanged due to the stand column assembly 301 being stationary. As such, the X-ray generation mechanism 201 cannot rotate too much relative to the subject to be imaged, otherwise the X-ray detector cannot normally receive X-rays, which causes an undesirable limitation on the three-dimensional imaging (tomographic imaging) of the subject to be imaged. For this reason, in some X-ray imaging systems, the above limitation is overcome by rotating subjects to be imaged, but this may cause safety problems such as the subjects to be imaged falling, and may also cause poor imaging effects due to shaking of the subjects to be imaged during the rotation.

### SUMMARY OF THE INVENTION

Based on the above problems in the prior art, one object of the present application is to provide a stand column assembly for an X-ray imaging system, which can avoid the safety problems that may arise from rotating a subject to be imaged during imaging, and can also avoid the adverse interference caused by the rotation of the subject to be imaged on the imaging effect.

Another object of the present application is to provide an X-ray imaging system comprising the above-mentioned stand column assembly.

To achieve the foregoing objects, the implementations of the present application may adopt the following technical solutions.

The implementations of the present application provide a stand column assembly for an X-ray imaging system as follows, comprising:
a base, configured to be disposed on a horizontal supporting surface and stationary relative to the supporting surface, the base being configured to support a subject to be imaged;
and

a stand column, comprising a main body portion and an X-ray detector, the main body portion being rotatably connected to the base and being erected opposite the supporting surface, and the X-ray detector being mounted on the main body portion, the main body portion being rotatable relative to the base within a predetermined range around an axis of rotation that is perpendicular to the supporting surface and passes through the base.

In an alternative solution, the stand column assembly further comprises a stand column rotation mechanism comprising:
a first power source, mounted on the base;
a driving gear, drivingly coupled to the first power source;
a turntable, serving as a driven gear constantly engaged with the driving gear, the turntable being rotatably mounted on the base, the turntable being capable of being driven to rotate by the first power source via the driving gear, and a central axis of the turntable serving as the axis of rotation; and
a support seat, fixedly mounted on the turntable and having an extension portion extending to the outside of the base, the main body portion being mounted on the extension portion.

In another alternative solution, the stand column assembly further comprises a first sensing assembly comprising at least one first sensor and a first triggering portion configured to trigger the first sensor,
one of the at least one first sensor and the first triggering portion being mounted on the support seat, and the other of the at least one first sensor and the first triggering portion being mounted on the base.

In another alternative solution, the sensing assembly comprises a plurality of said first sensors and one said first triggering portion, the plurality of said first sensors are arranged on a same circle with a center at the axis of rotation, and the one said first triggering portion when located at different positions can trigger different said first sensors.

In another alternative solution, the stand column assembly further comprises a stand column translation mechanism mounted on the extension portion and configured to drive the stand column to reciprocate linearly within a predetermined range.

In another alternative solution, the stand column rotation mechanism comprises:
a second power source;
a lead screw, extending linearly along a radial direction of the turntable, the lead screw being drivingly coupled to the second power source, and the lead screw being capable of being driven by the second power source to rotate relative to the extension portion; and
a nut, sleeved on the lead screw and threadedly coupled to the lead screw, the nut being fixed to the main body portion such that the lead screw can be driven by the second power source to drive the nut to move, thereby enabling the stand column to reciprocate linearly within the predetermined range.

In another alternative solution, the stand column assembly further comprises a second sensing assembly comprising at least one second sensor and a second triggering portion configured to trigger the second sensor,
one of the at least one second sensor and the second triggering portion being mounted on the extension portion, and the other of the at least one second sensor and the second triggering portion being mounted on the main body portion.

In another alternative solution, the stand column assembly further comprises a shielding portion, the shielding portion being detachably mounted on the base, and the shielding portion being translatable relative to the base and fixed relative to the base after being translated into position,
the shielding portion being positionable between the subject to be imaged and the X-ray detector in a state where the subject to be imaged is supported on the base.

In another alternative solution, the base has a support portion and a slide rail, the support portion being configured to support the subject to be imaged, and the slide rail being disposed on the top of the support portion;
a groove that fits with the slide rail is formed at the bottom of the shielding portion, and the slide rail is inserted into the groove so that the shielding portion is translatable along the slide rail;
and the stand column assembly further comprises a positioning member, the positioning member being mounted on the shielding portion, and the positioning member enabling the shielding portion, after being translated into position, to be fixed to the base.

In another alternative solution, the stand column assembly further comprises a positioning sensing assembly, the positioning sensing assembly being mounted on the shielding portion and/or the base,
and the positioning sensing assembly comprising a first multi-line laser radar and a second multi-line laser radar, scanning lines of the first multi-line laser radar lying in a first scanning plane, scanning lines of the second multi-line laser radar lying in a second scanning plane, the first scanning plane and the second scanning plane being orthogonal to each other.

The present application further provides an X-ray imaging system as follows, comprising:
the stand column assembly for an X-ray imaging system according to any one of the above technical solutions; and
an X-ray collimator, capable of synchronously rotating with the X-ray detector of the stand column assembly,
in a state where the subject to be imaged is supported on the base, the X-ray collimator and the X-ray detector being capable of rotating around the subject to be imaged, and the subject to be imaged continuously being located between the X-ray collimator and the X-ray detector.

### BRIEF DESCRIPTION OF THE DRAWINGS

FIG. 1 is a schematic side view showing a partial configuration of an X-ray imaging system according to an embodiment of the present application, in which the X-ray imaging system performs three-dimensional imaging on a subject to be imaged, such as a patient.

FIG. 2 is a schematic top view showing the partial configuration of the X-ray imaging system in FIG. 1.

FIG. 3 is an explanatory diagram showing a use state of the X-ray imaging system in FIG. 1, in which an X-ray detector and an X-ray collimator are in a first synchronous position.

FIG. 4 is an explanatory diagram showing a use state of the X-ray imaging system in FIG. 1, in which the X-ray detector and the X-ray collimator are in a second synchronous position.

FIG. 5 is an explanatory diagram showing a use state of the X-ray imaging system in FIG. 1, in which the X-ray detector and the X-ray collimator are in a third synchronous position.

FIG. 6 is schematic perspective view illustrating a stand column assembly of the X-ray imaging system in FIG. 1.

FIG. 7 is another schematic perspective view illustrating the stand column assembly in FIG. 6.

FIG. 8 is an explanatory diagram showing a use state of the stand column assembly in FIG. 6, in which the stand column is in a first translation position.

FIG. 9 is an explanatory diagram showing a use state of the stand column assembly in FIG. 6, in which the stand column is in a second translational position different from the first translational position.

FIG. 10 is a schematic perspective view illustrating a partial structure of the stand column assembly in FIG. 6.

FIG. 11 is a schematic perspective view showing a partial structure of the stand column assembly in FIG. 6, in which structures of a bottom of a stand column, a bottom of a shielding portion, a stand column rotation mechanism and a stand column translation mechanism are mainly shown.

FIG. 12 is a schematic perspective view showing a partial structure of the stand column assembly in FIG. 6, in which the structures of the stand column rotation mechanism and the stand column translation mechanism are mainly shown.

FIGS. 13 to 15 are schematic perspective views showing the stand column rotation mechanism of the stand column assembly in FIG. 6.

FIGS. 16 and 17 are schematic perspective views showing the stand column translation mechanism of the stand column assembly in FIG. 6.

FIG. 18 is an explanatory diagram showing a use state of the stand column translation mechanism in FIGS. 16 and 17, in which the stand column is in a first translation position.

FIG. 19 is an explanatory diagram showing a use state of the stand column translation mechanism in FIGS. 16 and 17, in which the stand column is in a second translation position.

FIG. 20 is a schematic perspective view showing a shielding portion of the stand column assembly in FIG. 6.

FIG. 21 is a schematic diagram showing a partial structure of the shielding portion in FIG. 20, in which a groove of the shielding portion is mainly shown.

FIG. 22 is a schematic perspective view showing a partial structure of a base of the stand column assembly in FIG. 6, in which a slide rail of the base is mainly shown.

FIGS. 23 and 24 are explanatory diagrams showing an assembled state of the groove of the shielding portion in FIG. 21 and the slide rail of the base in FIG. 22.

FIGS. 25 and 26 are explanatory diagrams showing a use state of a positioning sensing assembly of the stand column assembly in FIG. 6.

FIG. 27 is a schematic perspective view showing an existing X-ray imaging system.

### DETAILED DESCRIPTION

Embodiments of the present application are described below with reference to the accompanying drawings. For ease of understanding, elements shown in the drawings may include elements whose dimensions, scales and the like differ from actual dimensions, scales, and the like. Additionally, in order to provide a concise description in the specific description process of the embodiments, not all features of the embodiments are described in detail. For those of ordinary skill in the art related to the disclosure of the present application, some supplements and refinements, as well as design, manufacture, or production changes made on the basis of the technical content disclosed in the present application are common technical means and still fall within the scope of the present application, and should not be construed as the disclosure of the present application being insufficient.

Unless defined otherwise, the technical terms or scientific terms used in the claims and the description should have the usual meanings that are understood by those skilled in the art to which the present application belongs. Terms such as “first”, “second”, and similar terms used in the description and claims of the present application do not denote any order, quantity, or importance, but are only intended to distinguish different constituents. The word “include,” “comprise,” or a similar word is intended to mean that a component or an object that appears before “include” or “comprise” encompasses a component or an object and equivalent components that are listed after “include” or “comprise,” and does not exclude other components or objects. The terms “connect” or “link” and similar words are not limited to physical or mechanical connections, and are not limited to direct or indirect connections.

In the present application, “driving couple” between two components refers to that the two components are connected to each other in a manner capable of transmitting torque, including direct and indirect connections. A “torsion-resistant” connection achieved between two components refers to that the two components are connected such that they basically cannot rotate relative to each other.

The structure of an X-ray imaging system according to the embodiments of the present application, especially the structure of a stand column assembly of the X-ray imaging system, is described below with reference to the accompanying drawings.

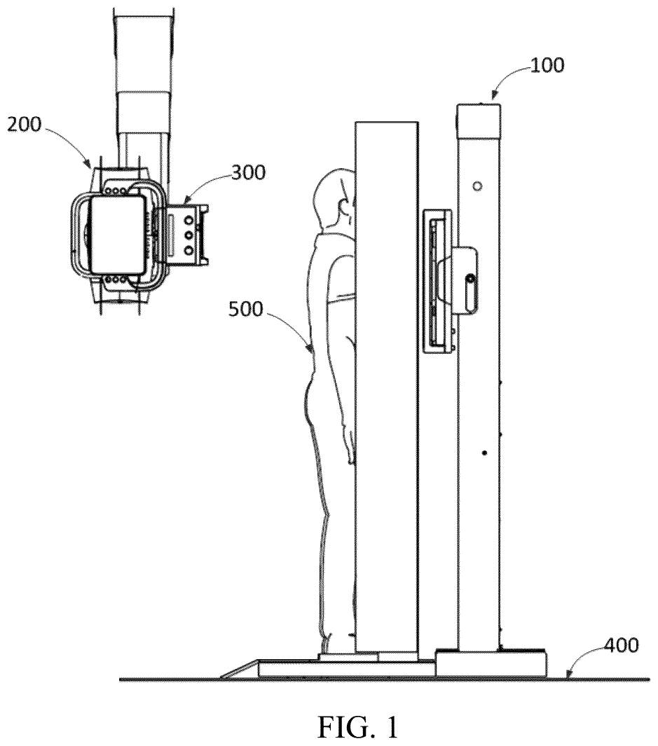

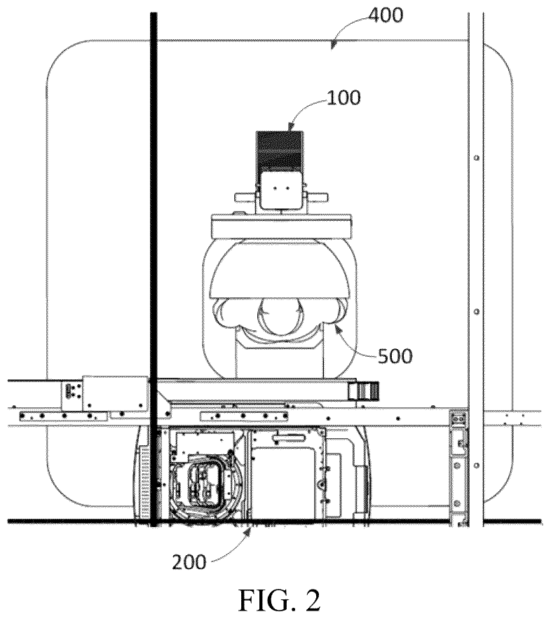

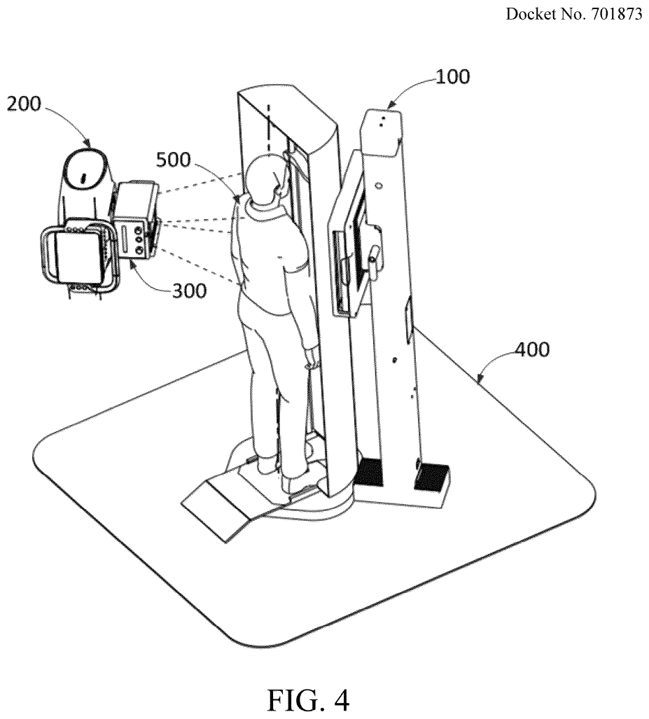

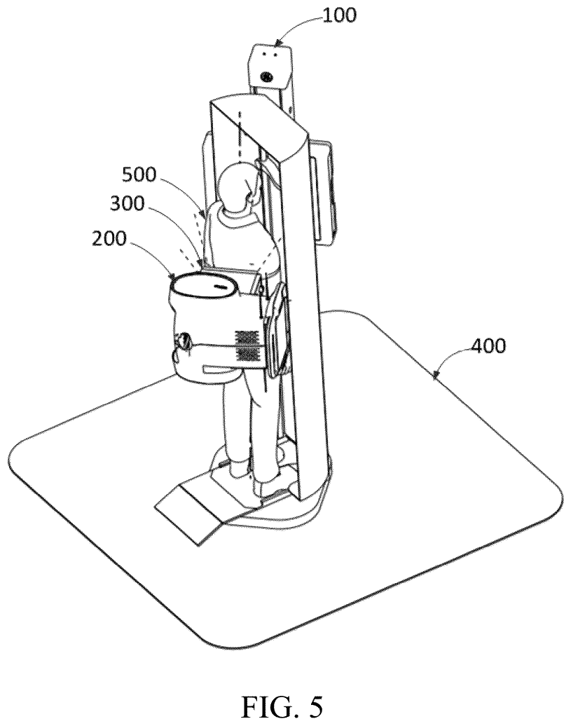

As shown in FIGS. 1 to 5, the X-ray imaging system according to the embodiments of the present application may include a stand column assembly 100, an X-ray generator (tube generator) 200, and an X-ray collimator 300.

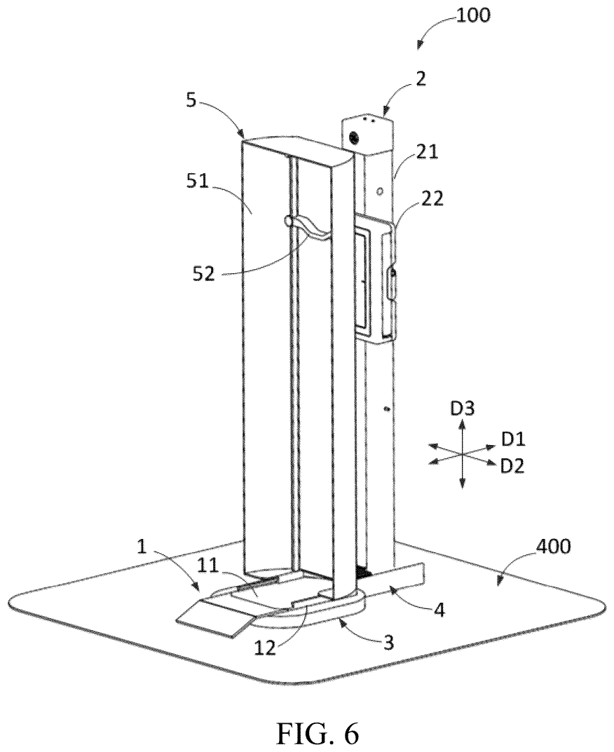

The stand column assembly 100 may be mounted on a horizontal mounting surface, e.g., a floor of a building, etc. Herein, “horizontal” includes not only completely horizontal but also substantially horizontal. The term “substantially” means that within a reasonable error range (e.g., an angle of 10 degrees or less is formed between the mounting surface and the horizontal plane) recognized by those skilled in the art, it can be considered that the mounting surface is a horizontal mounting surface. Referring to FIG. 6, the stand column assembly 100 includes structures such as a base 1 and a stand column 2 assembled together, and the stand column 2 is provided with an X-ray detector 22. The specific structure of each part of the stand column assembly 100 will be described in detail in the following.

The X-ray generator 200 may include a tube assembly capable of generating X-rays. The X-ray collimator 300 confines the X-rays generated by the X-ray generator 200 within a predetermined range, thereby enabling the X-rays to be concentrated at a predetermined site of a subject to be imaged 500. The present application does not limit the specific configuration of the X-ray generator 200, and the X-ray generator 200 may use various technologies that are present or will appear in the future. The X-ray generator 200 and the X-ray collimator 300 may be mounted on, for example, a ceiling (an indoor top surface) of a building or the like via a suspension apparatus, and the suspension apparatus can hold the X-ray generator 200 and the X-ray collimator 300 and adjust the positions and postures of the X-ray generator 200 and the X-ray collimator 300.

It is understandable that the suspension apparatus may also be mounted on a wall surface or a bracket, etc. The suspension apparatus may include a main body frame, a telescopic cylinder, and an angle adjustment mechanism that are assembled together. The main body frame holds and supports the telescopic cylinder and the angle adjustment mechanism, the main body frame may include a rail mechanism composed of a plurality of rails, and the plurality of rails of the rail mechanism can guide the main body frame to translate within a predetermined plane, whereby the main body frame can be positioned at any position within a predetermined range in the predetermined plane. The telescopic cylinder may have a plurality of sleeves that are coaxially arranged, each sleeve may have a different diameter, and the sleeves are nested together to form a controllable telescopic configuration. One end portion of the telescopic cylinder is mounted on the main body frame and the other end portion is connected to the angle adjustment mechanism, and a mechanism capable of driving the angle adjustment mechanism to rotate around a central axis of the telescopic cylinder may be provided inside the telescopic cylinder, such that the telescopic cylinder can not only drive the angle adjustment mechanism to reciprocate in a vertical direction perpendicular to a horizontal plane serving as the predetermined plane, but also drive the angle adjustment mechanism to rotate around the central axis of the telescopic cylinder. Furthermore, the angle adjustment mechanism is further connected to the X-ray generator 200 and the X-ray collimator 300, and is primarily configured to adjust rotation angles of the X-ray generator 200 and the X-ray collimator 300 rotating about an axis extending in a horizontal direction. Thus, the suspension apparatus can move in a predetermined range in multiple spatial degrees of freedom, thereby driving the X-ray generator 200 and the X-ray collimator 300 to move correspondingly.

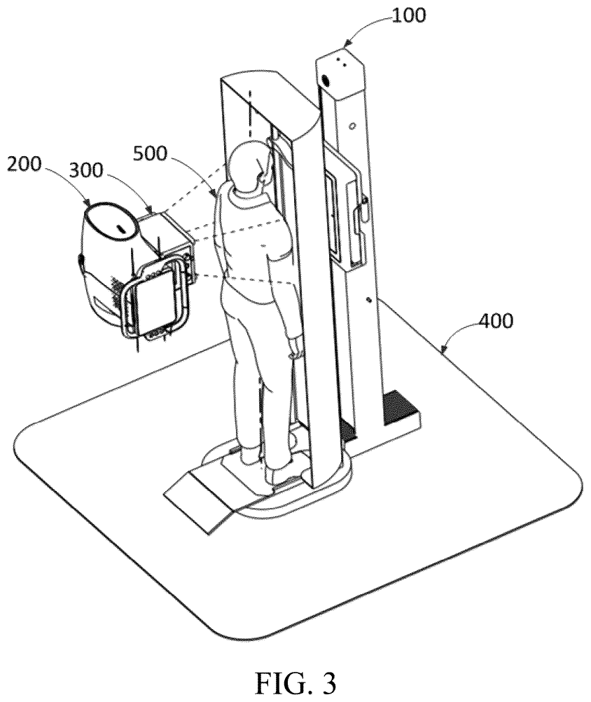

After the subject to be imaged 500, such as a patient, stands on the base 1 of the stand column assembly 100, the subject to be imaged 500 may remain stationary, and under the control of a control unit, the X-ray generator 200 and the X-ray collimator 300, as well as the stand column 2 and the X-ray detector 22 of the stand column assembly 100, can synchronously rotate around the subject to be imaged 500 to desired synchronous positions (see FIGS. 3 to 5), so that the X-ray imaging system can be utilized to perform desired three-dimensional imaging on the subject to be imaged 500.

The specific configuration of the stand column assembly 100 will be specifically described below.

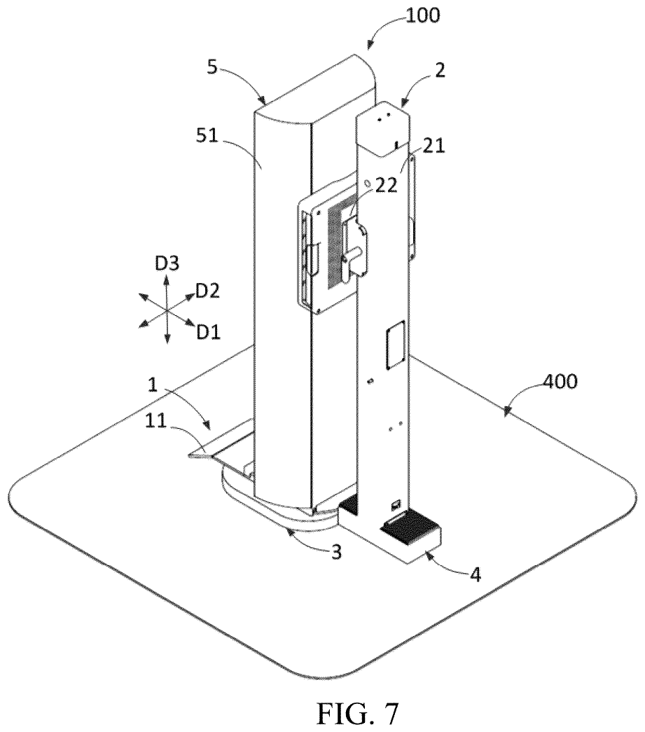

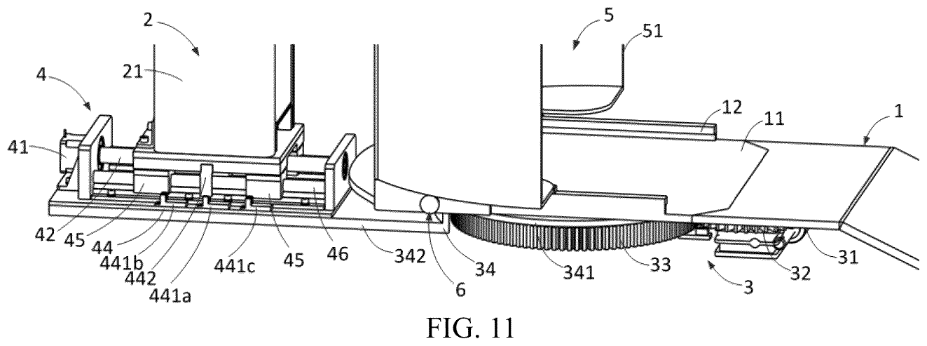

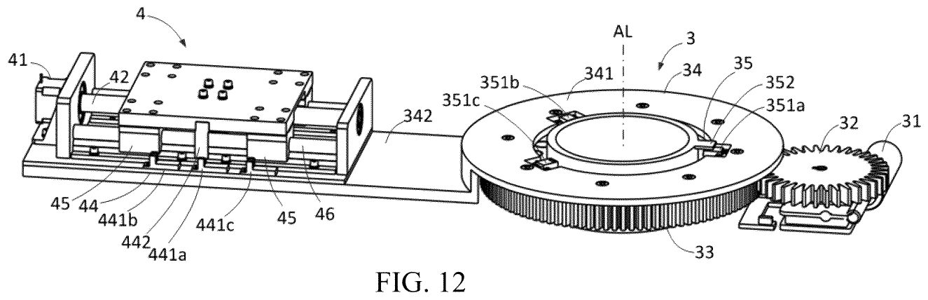

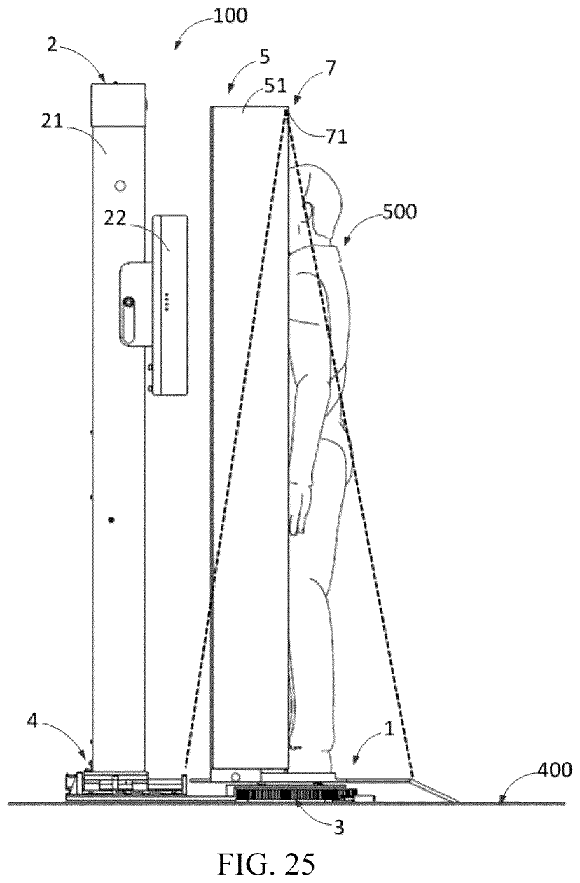

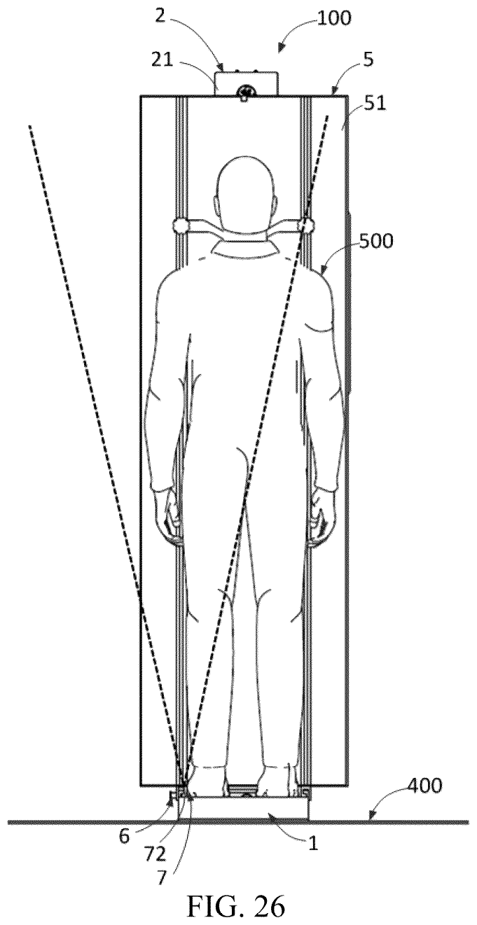

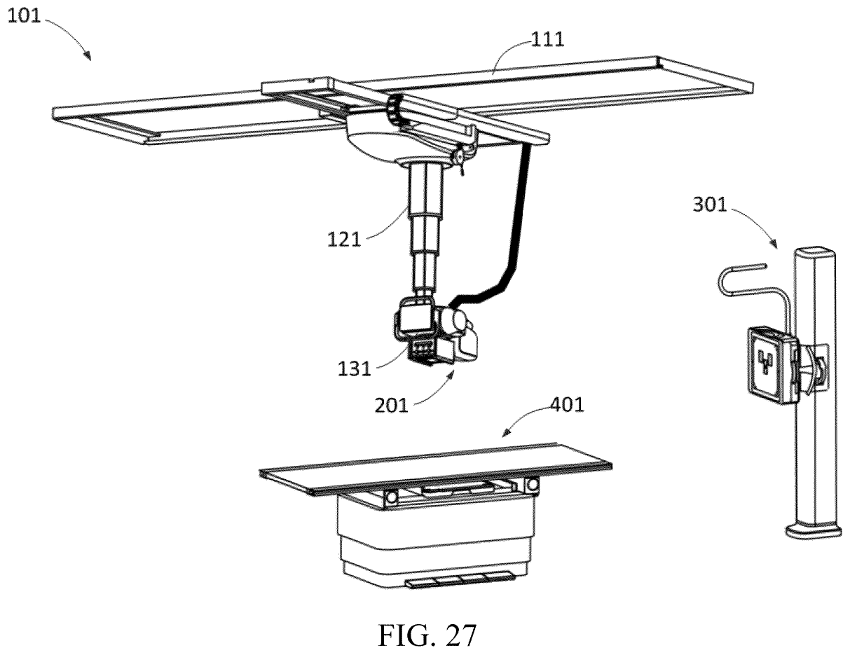

In the present embodiment, as shown in FIGS. 6 to 9, the stand column assembly 100 according to the embodiments of the present application includes a base 1, a stand column 2, a stand column rotation mechanism 3, a first sensing assembly 35 (see, for example, FIG. 12), a stand column translation mechanism 4, a second sensing assembly 44 (see, for example, FIGS. 11 and 12), a shielding portion 5, a positioning member 6, and a positioning sensing assembly 7 (see, for example, FIGS. 25 and 26) that are assembled together. For convenience of explanation, a front-rear direction D1, a left-right direction D2, and an up-down direction D3 below are adopted. Specifically, when the stand column assembly 100 is in an initial state as shown in FIGS. 6 and 7, the stand column 2 and the base 1 are arranged in the front-rear direction D1 and the stand column 2 is located in front of the base 1, and in a horizontal supporting surface 400 for disposing the base 1, a direction perpendicular to the front-rear direction D1 is the left-right direction D2, and a direction perpendicular to both the front-rear direction D1 and the left-right direction D2 is the up-down direction D3.

Specifically, in the present embodiment, as shown in FIGS. 6 to 9, the base 1 is disposed on the horizontal supporting surface 400 and stationary relative to the supporting surface 400, and the base 1 is configured to support the subject to be imaged 500. The base 1 may include a support portion 11 and two slide rails 12 fixed to each other. The support portion 11 is fixed to the supporting surface 400 and stably supported by the supporting surface 400. A top surface of the support portion 11 is formed to be parallel to the supporting surface 400 and has a sufficient space for the subject to be imaged 500 to stand, so that the subject to be imaged 500 can actively or passively move to the top surface of the support portion 11 and stand in place. The two slide rails 12 are fixedly disposed on the top of the support portion 11, and the shielding portion 5 can be supported by the slide rails 12 and slide translationally relative to the base 1 in the front-rear direction D1 along the slide rails 12. The two slide rails 12 are erected from the top surface of the support portion 11, and the two slide rails 12 are parallel to each other and extend linearly along the front-rear direction D1. Furthermore, the two slide rails 12 are spaced apart by a sufficiently large distance in the left-right direction D2, so that a sufficient space is left between the two slide rails 12 for the subject to be imaged 500 to stand. Further, three positioning holes penetrating in the left-right direction D2 may be formed on one slide rail 12, and a predetermined distance is provided between adjacent positioning holes. By engaging the positioning member 6 inserted into the shielding portion 5 with different positioning holes, the shielding portion 5 can be positioned at different positions relative to the base 1.

It is understandable that the shape and size of the support portion 11 may be adjusted as needed, provided that the support portion 11 can be stably supported by the supporting surface 400, and consequently the support portion 11 can stably support the subject to be imaged 500. Additionally, the number of the positioning hole on the slide rail 12 and the distance between adjacent positioning holes can be selected as required. Moreover, to facilitate mutual fixation between the base 1 and the shielding portion 5, the positioning holes may be formed as threaded holes, and accordingly the positioning member 6 may be formed as a threaded member fitting with the positioning holes.

In the present embodiment, as shown in FIGS. 6 to 9, the stand column 2 includes a main body portion 21 and an X-ray detector 22. The main body portion 21 is formed as a rod-like shape extending linearly along the up-down direction D3, and an interior of the main body portion 21 may be formed as a hollow configuration for mounting other components such as a circuit board. The main body portion 21 is also rotatably connected to the base 1 and erected opposite the supporting surface 400, the main body portion 21 can be driven by the stand column rotation mechanism 3 to rotate around the subject to be imaged 500 within a predetermined range, and the main body portion 21 can be driven by the stand column translation mechanism 4 to translate relative to the subject to be imaged 500 within a predetermined range. Further, the X-ray detector 22 may be fixedly mounted on the main body portion 21 for detecting X-rays penetrating the subject to be imaged 500.

It is understandable that, according to requirements of imaging different sites of the subject to be imaged 500, the X-ray detector 22 may be fixedly disposed at an appropriate position of the main body portion 21, for example, in the present embodiment, to image an oral cavity site of the subject to be imaged 500, the X-ray detector 22 is disposed at a position corresponding to the oral cavity of the subject to be imaged 500 in the up-down direction D3 of the main body portion 21. In addition, the X-ray detector 22 may also be configured to be translatable relative to the main body portion 21 along the up-down direction D3.

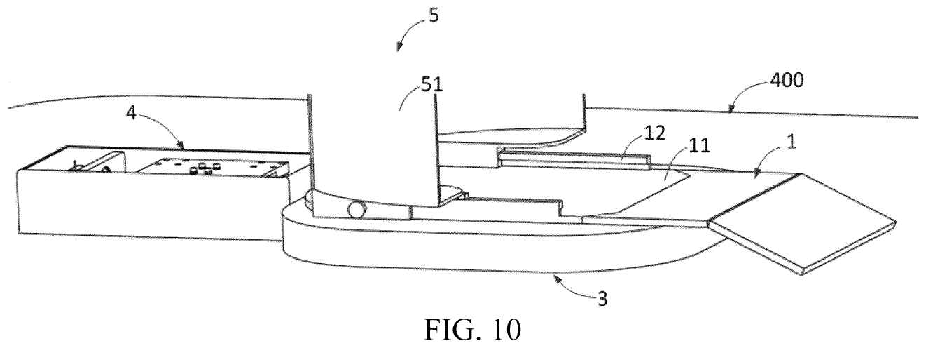

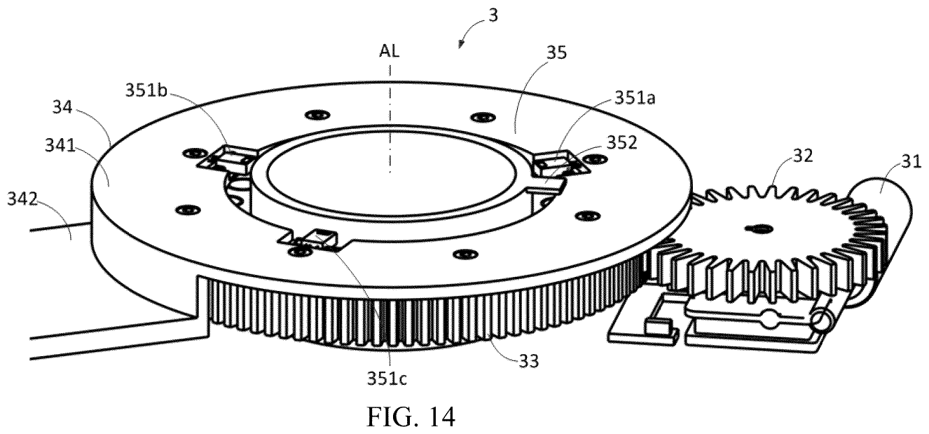

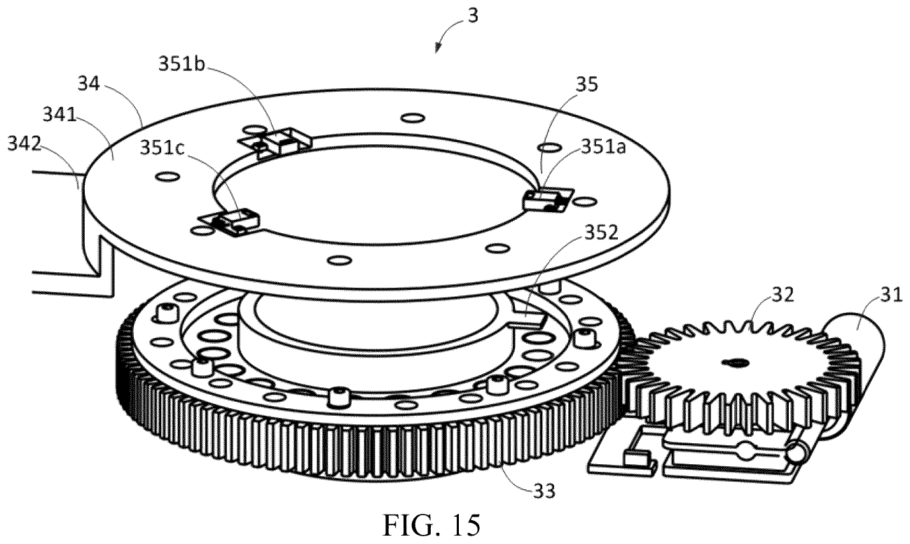

In the present embodiment, as shown in FIGS. 3 to 5, the stand column rotation mechanism 3 can drive the stand column 2 (the main body portion 21) to rotate relative to the base 1 about a predetermined axis of rotation AL (see, for example, FIG. 12), and the axis of rotation AL is perpendicular to the supporting surface 400 and passes through the base 1. The axis of rotation AL also passes through the subject to be imaged 500 in a state where the subject to be imaged is disposed on the base 1, whereby the stand column 2 can be rotated to a desired position relative to the base 1 (the subject to be imaged 500). Further, as shown in FIGS. 10 to 15, the stand column rotation mechanism 3 includes a first power source 31, a driving gear 32, a turntable 33 and a support seat 34, where the entirety of the first power source 31, the driving gear 32 and the turntable 33 and a part of the support seat 34 can be accommodated inside the base 1, so that the space occupied by the stand column assembly 100 can be saved, and the stand column rotation mechanism 3 can be prevented from being undesirably interfered during the operation and any adverse impact on the safety of the subject to be imaged 500 caused by the stand column rotation mechanism 3 can be prevented.

The first power source 31 may be a motor. The motor may include a stator, a rotor and a motor shaft that are coaxially assembled together, and the rotor and the motor shaft may be connected in a torsion-resistant manner and rotate relative to the stator. When the motor is powered on and in an operating state, the rotor can rotate relative to the stator, and then a torque is transmitted to the exterior of the motor through the motor shaft. The first power source 31 is in the form of a motor, which can reduce the space occupied by the power source and obtain a stable torque output. As shown in FIGS. 11 to 15, the motor shaft of the motor is drivingly coupled to the driving gear 32, so that the torque from the motor can be transmitted to the driving gear 32, thereby driving the driving gear 32 to rotate.

As shown in FIGS. 11 to 15, the driving gear 32 is constantly drivingly coupled to the first power source 31 on the one hand, and is constantly externally engaged with the turntable 33 serving as a driven gear on the other hand. The turntable 33 is rotatably mounted on the base 1, so that the turntable 33 can be driven to rotate by the first power source 31 via the driving gear 32. The diameter of the driving gear 32 is less than the diameter of the turntable 33, so that the gear pair serves to increase the torque from the first power source 31, which can ensure that the stand column 2 is smoothly driven to rotate. A central axis of the driving gear 32 and a central axis of the turntable 33 both extend along the up-down direction D3, and the central axis of the turntable 33 serves as the above-mentioned axis of rotation AL.

As shown in FIGS. 11 to 15, the support seat 34 is fixedly mounted on the turntable 33. The support seat 34 has an annular base portion 341 directly fixedly mounted on the turntable 33, the base portion 341 is located above the turntable 33 and may be fixed to the top surface of the turntable 33 via a plurality of connecting members, and the base portion 341 is located inside the base 1. Additionally, the support seat 34 further has an extension portion 342, and the extension portion 342 is fixedly connected to the base portion 341 and extends from an outer peripheral edge of the base portion 341 to the outside of the base 1. The extension portion 342 is formed in a flat plate shape and is parallel to and spaced apart from the supporting surface 400. The extension portion 342 further has a sufficient mounting area such that the main body portion 21 of the stand column 2 can be mounted on the extension portion 342.

In this way, in the stand column rotation mechanism 3, rotation of the turntable 33 relative to the base 1 by means of gear drive is achieved, and then rotation of the stand column 2 relative to the base 1 can be achieved by a stable, reliable and relatively simple configuration. Moreover, by positioning the main body portion 21 of the stand column 2 on the extension portion 342 of the support seat 34, the stand column 2 does not cause undesired interference with other components such as the base 1 during the rotation process.

It is understandable that, in other alternative solutions, the first power source 31 may also adopt other forms of power sources, and a drive mechanism formed by the driving gear 32 and the turntable 33 may also adopt other forms of drive mechanisms such as a belt drive mechanism, provided that the first power source 31 can drive the support seat 34 to rotate, thereby driving the stand column 2 to rotate.

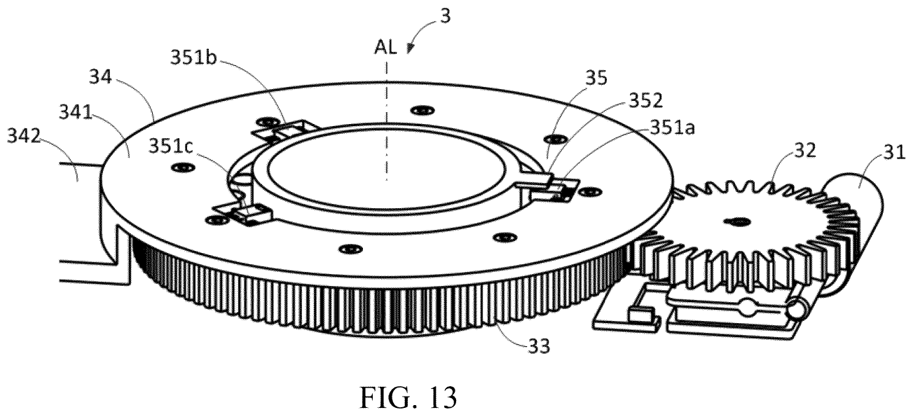

In the present embodiment, as shown in FIGS. 12 to 15, the first sensing assembly 35 includes three first sensors 351a, 351b, 351c and one first triggering portion 352. The three first sensors 351a, 351b, 351c are all fixedly mounted on a notch formed by an inner peripheral edge of the base portion 341 of the support seat 34, and respective geometric centers of the three first sensors 351a, 351b, 351c are arranged on a same circle with a center at the axis of rotation AL. The first triggering portion 352 may be fixedly mounted on the base 1, and the first triggering portion 352 is located above the base portion 341, that is, the first triggering portion 352 and the three first sensors 351a, 351b, 351c are staggered in the up-down direction D3. Only when the first sensors 351a, 351b, 351c rotate with the turntable 33 to a position where the first sensors 351a, 351b, 351c can sense the first triggering portion 352 (e.g., the first sensors 351a, 351b, 351c substantially overlap the first triggering portion 352 as viewed along the up-down direction D3), the corresponding first sensors can be triggered by the first triggering portion 352, and thus can send signals to the control unit. In this way, different first sensors 351a, 351b, and 351c can be triggered when the first triggering portion 352 is at different positions, thereby indicating different relative position relationships between the support seat 34 and the base 1. As shown in FIG. 13, triggering of the first sensor 351a on the right side of the figure indicates that the turntable 33 and the support seat 34 are at their initial positions, that is, the stand column 2 is at its initial position; and triggering of the two first sensors 351b, 351c on the left side of the figure indicates extreme positions for clockwise and counterclockwise rotations of the turntable 33 and the support seat 34, respectively (in the present embodiment, the extreme positions for clockwise and counterclockwise rotations rotate by 135 degrees of a central angle relative to the above initial positions), that is, the stand column 2 is at its extreme position.

In this way, the relative rotation positions of the support seat 34 and the turntable 33 relative to the base 1 can be determined using the first sensors 351a, 351b, 351c and the first triggering portion 352 that are mutually matched. Specifically, not only can the initial position of the stand column 2 be determined, but also the extreme position of the stand column 2 within the predetermined range can be determined, thereby facilitating the return of the stand column 2 to the initial state after work completion, and preventing excessive rotation of the stand column 2 during operation. Moreover, different first sensors 351a, 351b, 351c can be selectively triggered using one first triggering portion 352, so that different rotation positions of the stand column 2 are determined with a relatively simple configuration, and the structure of the entire stand column assembly 100 can be simplified.

It is understandable that, in other alternative solutions, the numbers of the first sensors 351a, 351b, 351c and the first triggering portion 352 may be selected as needed, provided that one of the first sensors 351a, 351b, 351c and the first triggering portion 352 is mounted on the support seat 34 or the turntable 33, and the other of the first sensors 351a, 351b, 351c and the first triggering portion 352 is mounted on the base 1. Additionally, the first sensors 351a, 351b, 351c may be Hall sensors, and the first trigger 352 may be a permanent magnet.

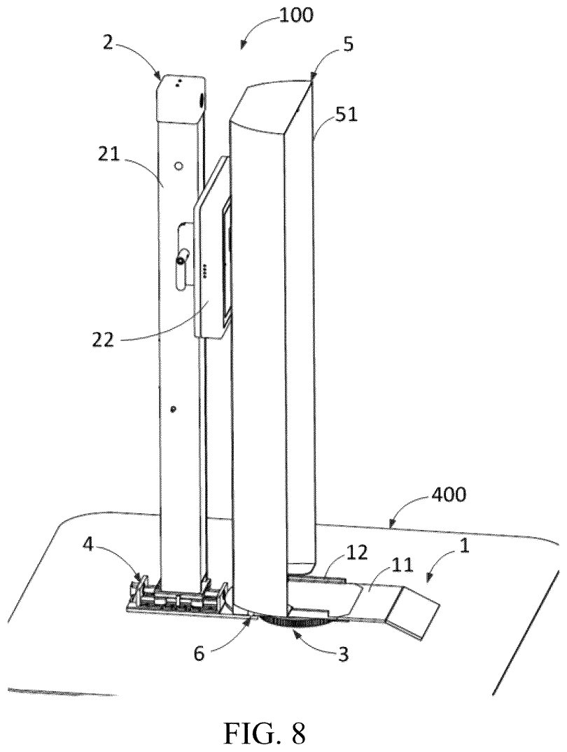

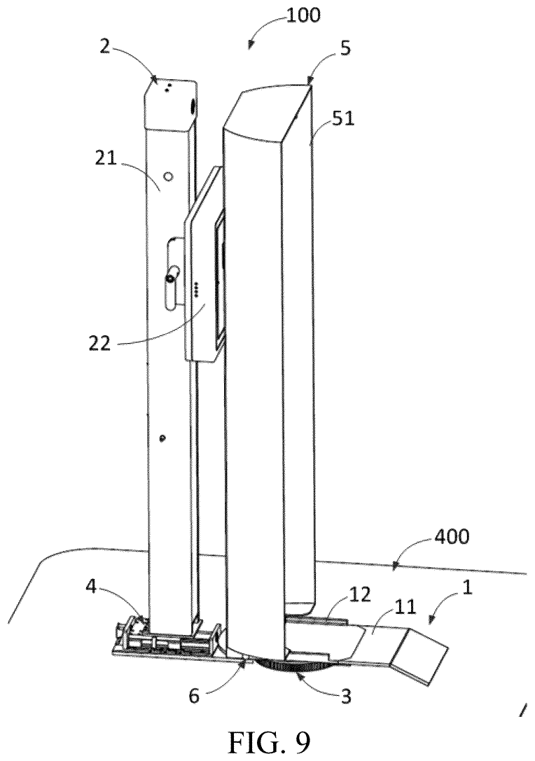

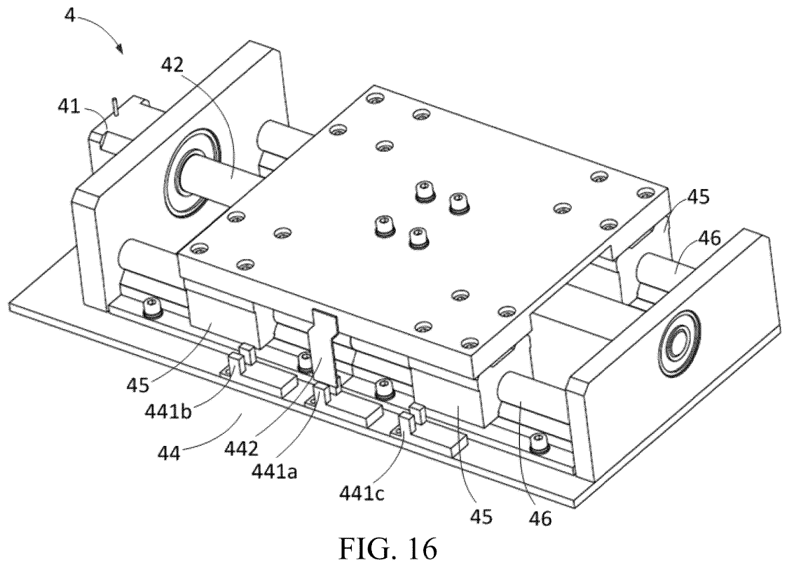

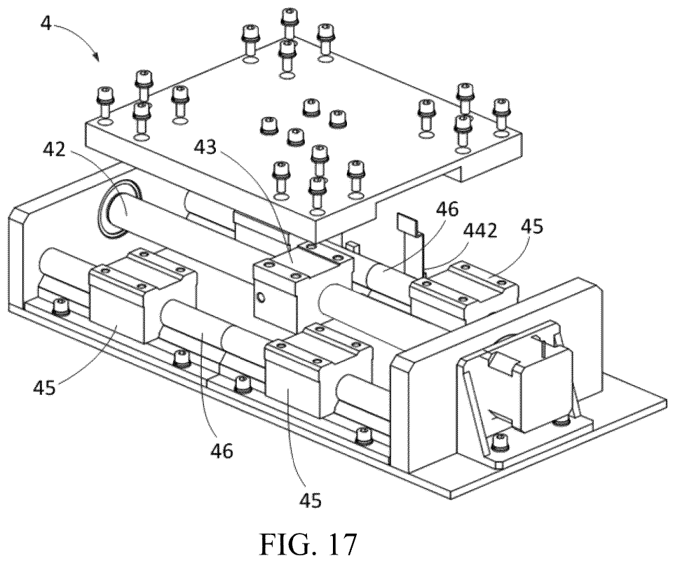

In the present embodiment, as shown in FIGS. 8 and 9, the stand column translation mechanism 4 can drive the stand column 2 (the main body portion 21) to translate relative to the base 1 along the front-rear direction D1, so that the stand column 2 can be translated to a desired position relative to the subject to be imaged 500. Further, as shown in FIGS. 16 to 19, the stand column translation mechanism 4 includes a second power source 41, a lead screw 42, a nut 43, guide blocks 45, and guide rails 46. The stand column translation mechanism 4 is entirely located below the stand column 2, and the stand column translation mechanism 4 is mounted on the extension portion 342 of the support seat 34 and can be driven by the support seat 34 to rotate together.

The second power source 41 may be a motor. The motor may include a stator, a rotor and a motor shaft that are coaxially assembled together, and the rotor and the motor shaft may be connected in a torsion-resistant manner and rotate relative to the stator. When the motor is powered on and in an operating state, the rotor can rotate relative to the stator, and then a torque is transmitted to the exterior of the motor through the motor shaft. The second power source 41 is in the form of a motor, which can reduce the space occupied by the power source and obtain a stable torque output. As shown in FIGS. 16 to 19, the motor shaft of the motor is drivingly coupled to the lead screw 42, so that the torque from the motor can be transmitted to the lead screw 42, thereby driving the lead screw 42 to rotate relative to the extension portion 342.

An external thread is formed on the lead screw 42, the lead screw 42 extends linearly along the radial direction of the turntable 33, and both ends of the lead screw 42 may be supported by bearing seats provided on the extension portion 342 via bearings. That is, when the stand column assembly 100 is in the initial state, the lead screw 42 extends linearly along the front-rear direction D1 and can rotate freely relative to the extension portion 342. An internal thread that is in screw-thread fit with the external thread of the lead screw 42 is formed on the nut 43, and the nut 43 is sleeved on the lead screw 42 and threadedly coupled to the lead screw 42. Furthermore, the nut 43 is fixed to the bottom of the main body portion 21 of the stand column 2, so that the lead screw 42 can be driven by the second power source 41 to drive the nut 43 to move, thereby enabling the stand column 2 to reciprocate linearly within a predetermined range.

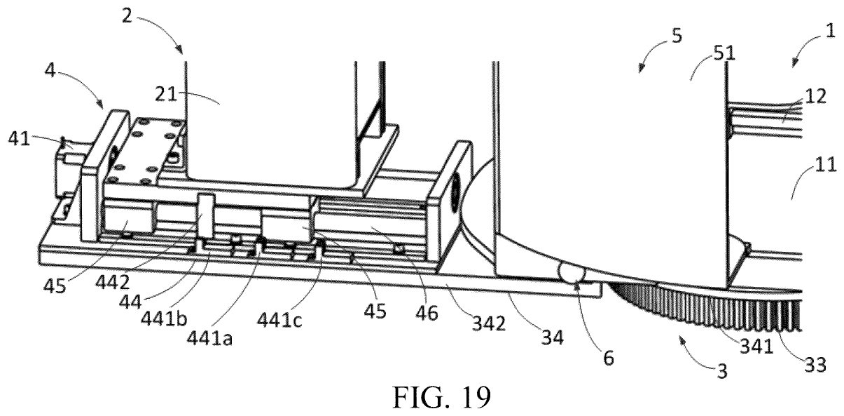

As shown in FIGS. 16 and 19, two guide rails 46 are provided on both sides of the lead screw 42, and each guide rail 46 is parallel to the lead screw 42 and fixed relative to the extension portion 342. Furthermore, two guide blocks 45 are provided on each guide rail 46, and a guide groove that is in shape fit with the guide rail 46 is formed on each guide block 45, so that the guide rail 46 can pass through the guide grooves of the guide blocks 45. All the guide blocks 45 are fixed to the bottom of the main body portion 21 of the stand column 2. Thus, during a reciprocating linear motion of the stand column 2, the guide blocks 45 and the guide rails 46 can prevent the stand column 2 from tilting during the motion.

In such a way, the stand column 2 can be driven by a lead screw nut mechanism to implement a reciprocating linear motion along the radial direction of the turntable 33, so that a radial distance between an X-ray receiver and the subject to be imaged 500 can be adjusted according to factors such as the position and bodily form of the subject to be imaged 500, thereby implementing desired three-dimensional imaging in collaboration with the stand column rotation mechanism 3.

It is understandable that, in other alternative solutions, the second power source 41 may also adopt other forms of power sources, and a drive mechanism formed by the lead screw 42 and the nut 43 may also adopt other forms of drive mechanisms such as a rack and pinion mechanism, provided that the second power source 41 can drive the stand column 2 to perform a reciprocating linear motion via the drive mechanism. Furthermore, through the stand column translation mechanism 4, a translation distance of the stand column 2 may be, for example, 50 mm or more, or may be other translation distances.

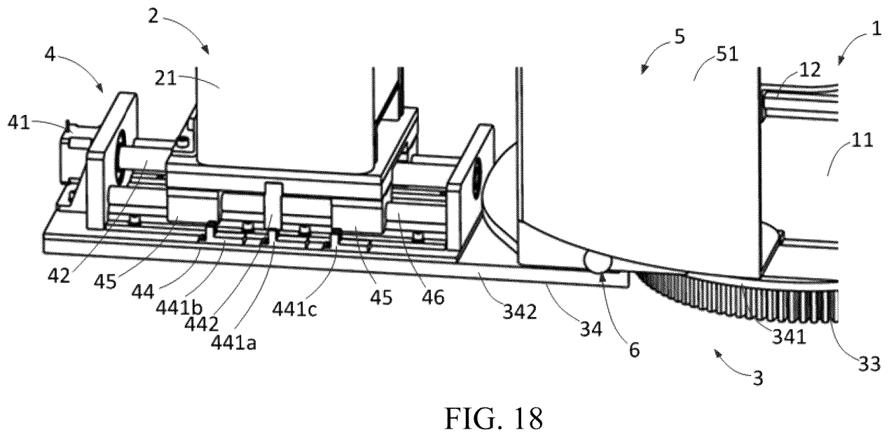

In the present embodiment, as shown in FIGS. 16 to 19, the second sensing assembly 44 includes three second sensors 441a, 441b, 441c and one second triggering portion 442. The three second sensors 441a, 441b, 441c are all fixedly mounted on the extension portion 342 of the support seat 34, and the three second sensors 441a, 441b, 441c are arranged at intervals in a direction parallel to the lead screw 42. The second triggering portion 442 may be fixedly mounted on the stand column 2. Only when the second triggering portion 442 is translated with the stand column 2 to a position where the second sensor 441a, 441b, 441c can be triggered, the corresponding second sensors 441a, 441b, 441c can be triggered by the second triggering portion 442, so that the triggered second sensor 441a, 441b, 441c can send signals to the control unit. In this way, different second sensors 441a, 441b, and 441c can be triggered when one second triggering portion 442 is at different positions, thereby indicating different relative position relationships between the stand column 2 and the support seat 34. As shown in FIG. 18 and FIG. 19, triggering of the second sensor 441a located in the middle indicates that the stand column 2 is at its initial position relative to the support seat 34, and triggering of the two second sensors 441b, 441c located on both sides indicates extreme positions of the reciprocating linear motion of the stand column 2 relative to the support seat 34.

In this way, a relative radial position of the stand column 2 relative to the base 1 can be determined by the second sensors 441a, 441b, 441c and the second triggering portion 442 that are mutually matched. In the case where the plurality of second sensors 441a, 441b, 441c are provided, not only the initial position of the stand column 2 but also the extreme position of the stand column 2 can be determined, thereby facilitating the return of the stand column 2 to the initial state after work completion, and preventing excessive translation of the stand column 2 during operation.

It is understandable that, in other alternative solutions, the numbers of the second sensors 441a, 441b, 441c and the second triggering portion 442 may be selected as needed, provided that one of the second sensors 441a, 441b, 441c and the second triggering portion 442 is mounted on the extension portion 342, and the other of the second sensors 441a, 441b, 441c and the second triggering portion 442 is mounted on the main body portion 21. Additionally, as an example, the second sensors 441a, 441b, and 441c may be light sensors, and the second trigger 442 may be a light shielding plate.

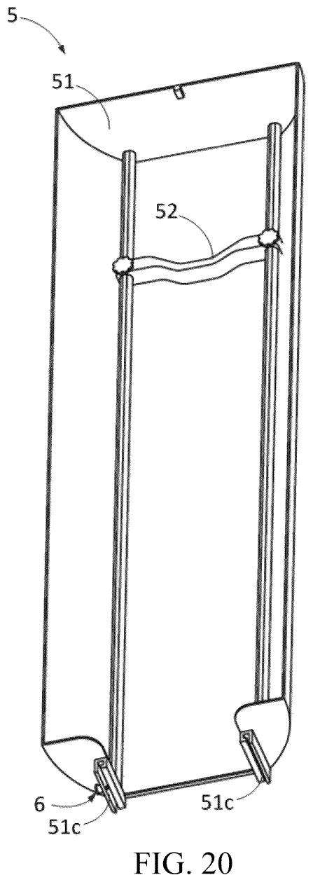

In the present embodiment, as shown in FIGS. 6 to 9, the shielding portion 5 is detachably mounted on the base 1, and the shielding portion 5 is translatable relative to the base 1 and fixed relative to the base 1 after being translated in place. Specifically, as shown in FIG. 20, the shielding portion 5 includes a hood portion 51 and a chin rest 52. In a state where the subject to be imaged 500 is supported on the base 1, the hood portion 51 of the shielding portion 5 is located between the subject to be imaged 500 and the X-ray detector 22. The chin rest 52 is provided at the hood portion 51, and the chin of the subject to be imaged 500 can be rested on the chin rest 52 after the subject to be imaged 500 stands in place, thereby facilitating accurate positioning of the oral cavity of the subject to be imaged 500, and further facilitating three-dimensional imaging of the oral cavity. Furthermore, the hood portion 51 may also be provided with two rails so that the position of the chin rest 52 can be adjusted in the up-down direction D3 along the two rails.

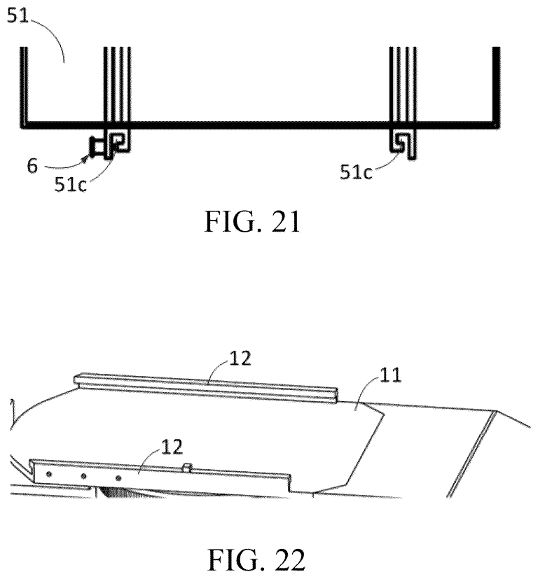

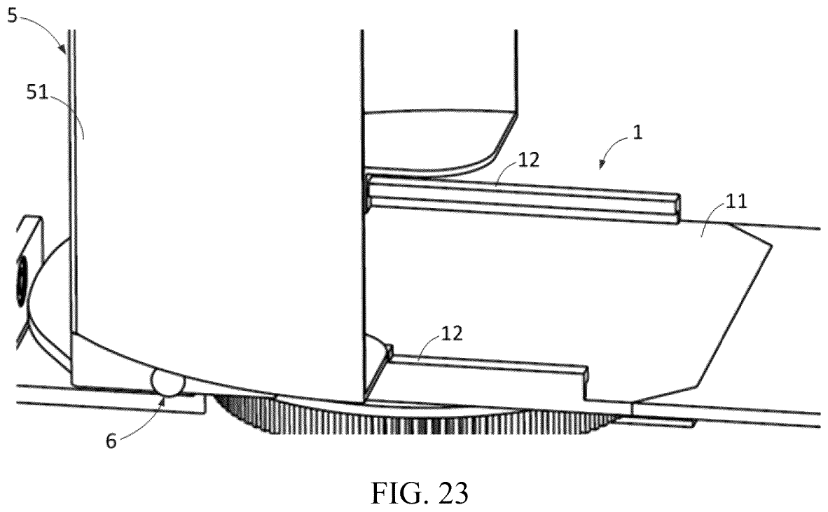

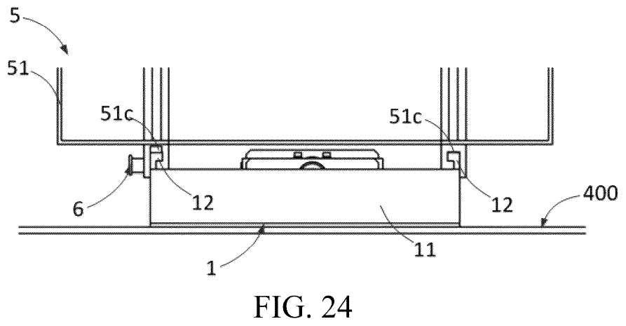

As shown in FIGS. 21 to 24, a groove 51c that fits with the slide rail 12 of the base 1 is formed at the bottom of the hood portion 51, and the slide rail 12 is inserted into the corresponding groove 51c, so that the shielding portion 5 can translate along the slide rail 12 in a state of being supported by the slide rail 12. As described above, the stand column assembly 100 further includes the positioning member 6, and the positioning member 6 is inserted through a corresponding mounting hole of the shielding portion 5. When the positioning member 6 is inserted into different positioning holes formed in the slide rail 12, the positioning member 6 can position the shielding portion 5 at different positions relative to the base 1, that is, the shielding portion 5 is fixed to the base 1 after being translated into position. In this way, use of the positioning member 6 can fix the shielding portion 5 to the base 1 after the shielding portion 5 is translated into position, thereby preventing the shielding portion 5 from moving undesirably after being translated into position.

In the case where the positioning of the positioning member 6 is released, for example, the positioning member 6 is not inserted into the positioning hole of the slide rail 12, or the positioning member 6 is removed from the shielding portion 5, the shielding portion 5 can be separated from the slide rail 12 after sliding along the slide rail 12.

In this way, the shielding portion 5 is detachably mounted on the base 1, so the stand column assembly 100 can operate in both states in which the shielding portion 5 is mounted and in which the shielding portion 5 is detached. In a state where the shielding portion 5 is provided, the shielding portion 5 can translate relative to the base 1 to perform position adjustment, which, on the one hand, can facilitate the positioning of the subject to be imaged 500 on the base 1, and on the other hand, in a case where the shielding portion 5 is provided with an additional portion such as the chin rest 52, can also facilitate the positioning of a specific site such as the chin of the subject to be imaged 500 on the additional portion, thereby facilitating the improvement of the imaging quality. Additionally, in a state where the shielding portion 5 is detached, an X-ray imaging system including the stand column assembly 100 according to the present application may be used to perform X-ray imaging on a site (e.g., a chest, a leg, or the like) of the subject to be imaged 500, such as a patient.

In the present embodiment, as shown in FIG. 25 and FIG. 26, the positioning sensing assembly 7 includes a first multi-line laser radar 71 and a second multi-line laser radar 72. The first multi-line laser radar 71 may be mounted on the top of the hood portion 51 of the shielding portion 5, and the second multi-line laser radar 72 may be mounted on the base 1. A plurality of scanning lines of the first multi-line laser radar 71 are located on a first scanning plane, and the first scanning plane may extend along the front-rear direction D1 and the up-down direction D3. A scanning line of the second multi-line laser radar 72 is located on a second scanning plane, and the second scanning line may extend in the left-right direction D2 and the up-down direction D3. Thereby, the first scanning plane and the second scanning plane are orthogonal to each other. In this way, the first multi-line laser radar 71 may be utilized to assist in positioning the subject to be imaged 500 in the front-rear direction D1, and the second multi-line laser radar 72 may be utilized to assist in positioning the subject to be imaged 500 in the left-right direction D2, thus enabling the subject to be imaged 500 to be positioned such that the axis of rotation AL passes through the subject to be imaged 500, and more advantageously the axis of rotation AL passes through the center of gravity of the subject to be imaged 500. Thus, it is beneficial for the X-ray imaging system to achieve more effective three-dimensional imaging of the subject to be imaged 500.

Since the scanning planes where the scanning lines of the two multi-line laser radars 71, 72 of the positioning sensing assembly 7 are located are orthogonal to each other, it is advantageous to use the scanning planes to determine the position of the subject to be imaged 500 in a two-dimensional coordinate system within the supporting surface of the base 1, so that the subject to be imaged 500 can be positioned as much as possible at a position where good three-dimensional imaging can be achieved.

It is understandable that, in other alternative solutions, the first scanning plane of the first multi-line laser radar 71 and the second scanning plane of the second multi-line laser radar 72 do not necessarily extend along the directions described in the above embodiment, provided that the two scanning planes are orthogonal to each other and accordingly can assist in positioning the subject to be imaged 500. In addition, the multi-line radars can be signal-connected to the control unit of the X-ray imaging system, whereby automatic positioning can be performed through the control unit. Additionally, the positioning sensing assembly 7 may include more multi-line laser radars, and these laser radars may be mounted at different positions of the shielding portion 5 and the base 1 as required, or even may be mounted on a wall or a ceiling of a building. In other alternative solutions, other sensors may be employed to assist in the positioning of the subject to be imaged 500.

Furthermore, the X-ray imaging system may include the control unit. In the present application, the stand column rotation mechanism 3, the stand column translation mechanism 4, the first sensing assembly 35, the second sensing assembly 44, the positioning sensing assembly 7, and the like may be signal-connected to the control unit, so that the control unit controls their operations.

By adopting the above technical solutions, in a case where three-dimensional imaging is required to be performed using the X-ray imaging system, the subject to be imaged 500 such as a patient may remain stationary, and the three-dimensional imaging is performed by synchronously rotating the stand column 2 provided with the X-ray detector 22 together with the X-ray collimator 300. In this way, both the safety problem that may arise from the rotation of the subject to be imaged 500 during imaging is avoided, and the undesirable movements such as shaking that may occur during the rotation of the subject to be imaged 500 and adverse interference to the imaging effect caused by factors such as occurrence of coordinate system transformation can be avoided.

It should be understood that the above embodiments are only exemplary and are not intended to limit the present application. Those skilled in the art may make various modifications and changes to the above embodiments under the teachings of the present disclosure, without departing from the scope of the present application. The technical solutions of the present application are supplementarily described as follows.
i. In the above specific embodiments, the stand column 2 can be rotated by 135 degrees clockwise and counterclockwise from the initial position using the stand column rotation mechanism 3, that is, the rotation range of the stand column 2 relative to the axis of rotation AL is 270 degrees, but the present application is not limited thereto. For example, the stand column 2 may be rotated by 360 degrees in a full circle using the stand column rotation mechanism 3, which is more advantageous for three-dimensional imaging by using the X-ray imaging system. Moreover, the rotation of the stand column 2 by the stand column rotation mechanism 3 not only improves the three-dimensional imaging capability compared to the X-ray imaging system fixed to the stand column 2, but also weakens the dependence of the three-dimensional imaging process on control methods such as algorithms.
ii. It is understandable that, the shielding portion 5 can adjust the position of the shielding portion 5 relative to the base 1 according to the bodily form of the subject to be imaged 500 and the position of the subject to be imaged 500. The chin of the subject to be imaged 500 can be positioned at a desired position by using the chin rest 52 of the shielding portion 5, which is beneficial to simplify the operation of the X-ray imaging system and simplify the work process.
iii. It is understandable that, the X-ray imaging system according to the present application can be used not only for three-dimensional imaging of the oral cavity of the subject to be imaged 500 and thus can be used for oral cavity condition monitoring and related applications, but also for three-dimensional imaging of other sites.

It should be understood that some of the various components, structures, and constituent parts described above may be omitted without affecting the achievement of one or more objects of the present application. Different embodiments, examples or aspects may be appropriately combined, provided that they do not conflict or contradict each other.

The exemplary embodiments and variants of the present application have been described above; however, it should be understood that various modifications may be made. For example, same, similar, or other suitable results can be achieved if the described techniques are executed in a different order and/or if components in the described systems, architectures, devices, or circuits are combined in different ways and/or replaced or supplemented by additional components or equivalents thereof; and these changes or modifications also fall within the scope of protection of the claims.

1. A stand column assembly for an X-ray imaging system, characterized by comprising:
a base, configured to be disposed on a horizontal supporting surface and stationary relative to the supporting surface, the base being configured to support a subject to be imaged; and
a stand column, comprising a main body portion and an X-ray detector, the main body portion being rotatably connected to the base and being erected opposite the supporting surface, the X-ray detector being mounted on the main body portion, the main body portion being rotatable relative to the base within a predetermined range around an axis of rotation that is perpendicular to the supporting surface and passes through the base.

2. The stand column assembly for an X-ray imaging system according to claim 1, further comprising a stand column rotation mechanism comprising:
a first power source, mounted on the base;
a driving gear, drivingly coupled to the first power source;
a turntable, serving as a driven gear constantly engaged with the driving gear, the turntable being rotatably mounted on the base, the turntable being capable of being driven to rotate by the first power source via the driving gear, and a central axis of the turntable serving as the axis of rotation; and
a support seat, fixedly mounted on the turntable and having an extension portion extending to the outside of the base, the main body portion being mounted on the extension portion.

3. The stand column assembly for an X-ray imaging system according to claim 2, further comprising a first sensing assembly comprising at least one first sensor and a first triggering portion configured to trigger the first sensor,
one of the at least one first sensor and the first triggering portion being mounted on the support seat, and the other of the at least one first sensor and the first triggering portion being mounted on the base.

4. The stand column assembly for an X-ray imaging system according to claim 3, wherein the sensing assembly comprises a plurality of said first sensors and one said first triggering portion, the plurality of said first sensors are arranged on a same circle with a center at the axis of rotation, and the one said first triggering portion when located at different positions can trigger different said first sensors.

5. The stand column assembly for an X-ray imaging system according to claim 2, further comprising a stand column translation mechanism mounted on the extension portion and configured to drive the stand column to reciprocate linearly within a predetermined range.

6. The stand column assembly for an X-ray imaging system according to claim 5, wherein the stand column translation mechanism comprises:
a second power source;
a lead screw, extending linearly along a radial direction of the turntable, the lead screw being drivingly coupled to the second power source, and the lead screw being capable of being driven by the second power source to rotate relative to the extension portion; and
a nut, sleeved on the lead screw and threadedly coupled to the lead screw, the nut being fixed to the main body portion such that the lead screw can be driven by the second power source to drive the nut to move, thereby enabling the stand column to reciprocate linearly within the predetermined range.

7. The stand column assembly for an X-ray imaging system according to claim 6, further comprising a second sensing assembly comprising at least one second sensor and a second triggering portion configured to trigger the second sensor,
one of the at least one second sensor and the second triggering portion being mounted on the extension portion, and the other of the at least one second sensor and the second triggering portion being mounted on the main body portion.

8. The stand column assembly for an X-ray imaging system according to claim 1, further comprising a shielding portion, the shielding portion being detachably mounted on the base, and the shielding portion being translatable relative to the base and fixed relative to the base after being translated into position,
the shielding portion being positionable between the subject to be imaged and the X-ray detector in a state where the subject to be imaged is supported on the base.

9. The stand column assembly for an X-ray imaging system according to claim 8, wherein the base has a support portion and a slide rail, the support portion being configured to support the subject to be imaged, and the slide rail being disposed on the top of the support portion;
a groove that fits with the slide rail is formed at the bottom of the shielding portion, and the slide rail is inserted into the groove so that the shielding portion is translatable along the slide rail;
and the stand column assembly further comprises a positioning member, the positioning member being mounted on the shielding portion, and the positioning member enabling the shielding portion, after being translated into position, to be fixed to the base.

10. The stand column assembly for an X-ray imaging system according to claim 8, further comprising a positioning sensing assembly, the positioning sensing assembly being mounted on the shielding portion and/or the base,
and the positioning sensing assembly comprising a first multi-line laser radar and a second multi-line laser radar, scanning lines of the first multi-line laser radar lying in a first scanning plane, scanning lines of the second multi-line laser radar lying in a second scanning plane, the first scanning plane and the second scanning plane being orthogonal to each other.

11. An X-ray imaging system, characterized by comprising:
a stand column assembly for an X-ray imaging system, characterized by comprising:
a base, configured to be disposed on a horizontal supporting surface and stationary relative to the supporting surface, the base being configured to support a subject to be imaged; and
a stand column, comprising a main body portion and an X-ray detector, the main body portion being rotatably connected to the base and being erected opposite the supporting surface, the X-ray detector being mounted on the main body portion, the main body portion being rotatable relative to the base within a predetermined range around an axis of rotation that is perpendicular to the supporting surface and passes through the base; and
an X-ray collimator, capable of synchronously rotating with the X-ray detector of the stand column assembly,
wherein in a state where the subject to be imaged is supported on the base, the X-ray collimator and the X-ray detector being capable of rotating around the subject to be imaged, and the subject to be imaged continuously being located between the X-ray collimator and the X-ray detector.
